(19) United States
(12) Patent Application Publication
(10) Pub. No.: US 2011/0022162 A1
(43) Pub. Date: Jan. 27, 2011

(54) ENDOPROSTHESES

(75) Inventors: Rajesh Radhakishnan, Maple Grove, MN (US); Liliana Atanasoska, Edina, MN (US); Daniel VanCamp, Elk River, MN (US)

Correspondence Address: FISH & RICHARDSON P.C. (BO), P.O. BOX 1022, MINNEAPOLIS, MN, 55440-1022, US

(73) Assignee: BOSTON SCIENTIFIC SCIMED, INC., Maple Grove, MN (US)

(21) Appl. No.: 12/508,092

(22) Filed: Jul. 23, 2009

(51) Int. Cl. A61F 2/06; B44C 1/22; C25D 11/00

(52) U.S. Cl. 623/1.46; 216/8; 205/106

(57) ABSTRACT

An endoprosthesis includes an endoprosthesis wall that includes a ceramic coating and a polymer coating containing a therapeutic agent. A ceramic coating can be formed on the wall by electrochemical deposition. Cyclic voltammetry can be conducted to modify the density of the hydroxyl groups on the surface of the ceramic coating.

### TECHNICAL FIELD

This invention relates to endoprostheses.

### BACKGROUND

The body includes various passageways such as arteries, other blood vessels and other body lumens. These passageways sometimes become occluded or weakened. For example, the passageways can be occluded by a tumor, restricted by plaque, or weakened by an aneurysm. When this occurs, the passageway can be reopened or reinforced with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, e.g., so that it can contact the walls of the lumen. Stent delivery is further discussed in Heath, U.S. Pat. No. 6,290,721.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

### SUMMARY

In one aspect, the invention features a method of making an endoprosthesis. The method includes electrochemically depositing a ceramic coating on a surface of an endoprosthesis wall.

In another aspect, the invention features a method of making an endoprosthesis. The method includes depositing a ceramic coating on a surface of an endoprosthesis wall and conducting cyclic voltammetry on the endoprosthesis wall.

In another aspect, the invention features a method of making an endoprosthesis. The method includes forming a metallic coating on a surface of an endoprosthesis wall and conducting cyclic voltammetry on the metallic coating to convert the metallic coating into a ceramic coating. The ceramic coating can include a hydroxylated surface.

In another aspect, the invention features an endoprosthesis preform including a ceramic coating. The ceramic coating includes surface hydroxyl groups that have a density of at least about 1.8×10⁻⁵ mol/m².

In another aspect, the invention features an endoprosthesis preform including a ceramic coating. The ceramic coating has an orange peel morphology and includes surface hydroxyl groups.

In another aspect, the invention features a method of treating an occlusion site in a vessel. The method includes providing a stent that has a polymer coating containing a therapeutic agent, the polymer coating being on a ceramic coating that includes surface hydroxyl groups having a density of at least 1.8×10⁻⁵ mol/m², accessing the site in the vessel with a catheter carrying the stent, expanding the stent to compress the occlusion, withdrawing the catheter from the vessel, and eluting the therapeutic agent from the stent.

In another aspect, the invention features a method of treating an occlusion site in a vessel. The method includes providing that has a polymer coating containing a therapeutic agent, the polymer coating being on a ceramic coating that includes surface hydroxyl groups having a density of at least 1.8×10⁻⁵ mol/m², accessing the site in the vessel with a catheter carrying the stent, expanding the stent to compress the occlusion, withdrawing the catheter from the vessel, and eluting the therapeutic agent from the stent.

Embodiments of the method of making an endoprosthesis may include any one or more of the following features. Cyclic voltammetry can be conducted on the endoprosthesis wall after depositing the ceramic coating. The surface can be a nano-structured surface. The nano-structured surface can be a surface of an endoprosthesis preform. The surface of the endoprosthesis preform can include abluminal, luminal, and cutface surfaces. The nano-structured surface can be formed by laser processing, ion bombardment, grit blasting, or electrolytic etching. The nano-structured surface can be formed by electrolytic etching. The nano-structured surface can be a surface of a coating between the ceramic coating and an endoprosthesis preform. A polymer coating can be formed on the ceramic coating. A tie layer can be formed between the polymer coating and the ceramic coating. The ceramic coating can include iridium oxide. The ceramic coating can include surface hydroxyl groups that have a density of at least 1.8×10⁻⁵ mol/m². The ceramic coating can be formed by depositing a metallic layer on the surface of the endoprosthesis wall. The metallic layer can be converted into a ceramic layer by, for example, applying a cyclic voltammetry to the metallic layer or by applying pulsed electrolytic waveforms to the metallic layer. The metallic layer can be deposited by applying a solution to the endoprosthesis wall. The solution can include an iridium hydrobromide acidic bath.

Embodiments of an endoprosthesis may include any one or more of the following features. The preform can include a nano-structured surface and the ceramic coating can be on the nano-structured surface. The coating can be between the ceramic coating and the preform. The coating can include a nano-structured surface and the ceramic coating can be on the nano-structured surface. A polymer coating can be on the ceramic coating. The polymer coating can include poly(lactic-co-glycolic acid). A tie layer can be between the ceramic coating and the polymer coating. The tie layer can include silane. The ceramic coating can include iridium oxide and can be conformally about the preform. The ceramic coating can have an orange peel morphology.

Embodiments and/or aspects may include any one or more of the following advantages. Endoprostheses can be provided that have an enhanced adhesion of a polymer coating that contains a therapeutic agent to a stent body (e.g. a metal). The nano-structured surface can provide mechanical interlocking to a ceramic coating on the stent body surface. The ceramic coating can be formed conformally about the stent body, e.g., by electrochemical deposition and have a low-roughness morphology, e.g., an orange peel morphology that has physiological benefits in reducing restenosis and enhancing endothalulyation on the adluminal surface region of the stent. Cyclic voltammetry can enhance the density of surface hydroxyl groups on the ceramic coating. The polymer coating can form chemical bonds with the surface hydroxyl groups and bond to the ceramic coating with enhanced adhesion. The tie layer can enhance the adhesion of the polymer coating to the stent body.

All publications, patent applications, patents, and other references mentioned herein are incorporated by reference herein in their entirety.

Other features and advantages of the invention will be apparent from the following detailed description, and from the claims.

### DESCRIPTION OF DRAWINGS

FIGS. 1A-1C are longitudinal cross-sectional views, illustrating delivery of a stent in a collapsed state, expansion of the stent, and deployment of the stent.

FIG. 2 is a perspective view of a fenestrated stent.

FIG. 3 is a cross-sectional view of a stent wall.

FIG. 3A is an enlarged plan view (photograph) of a part of a stent wall surface in FIG. 3.

FIGS. 4A-4C are an enlarged plan views (photographs) of a part of a stent wall surface of an over expanded stent.

FIG. 5A is an enlarged plan view (photograph) of an expanded and crimped stent.

FIG. 5B is an enlarged plan view (photograph) of a part of a stent wall surface in the crimped region of the stent in FIG. 5A.

FIG. 5C is an enlarged plan view (photograph) of another expanded and crimped stent.

FIG. 5D is an enlarged plan view (photograph) of a part of a stent wall surface in the crimped region of the stent in FIG. 5C.

FIGS. 6A-6C are diagrammatic representations of a method for making a stent in FIG. 3.

Like reference symbols in the various drawings indicate like elements.

### DETAILED DESCRIPTION

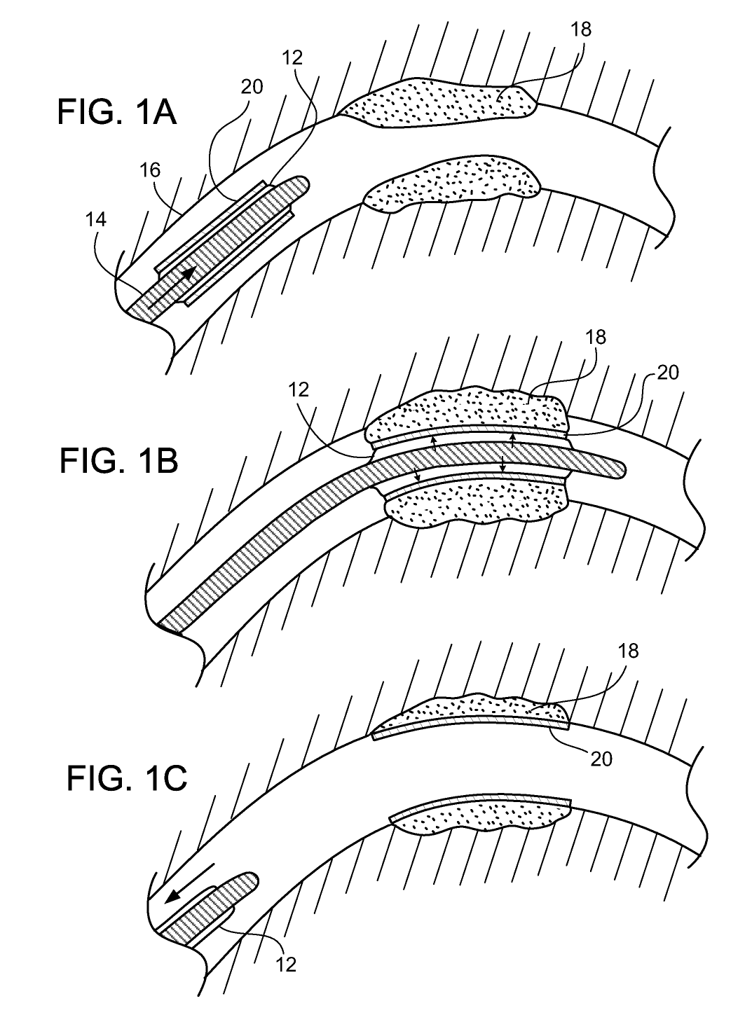

Referring to FIGS. 1A-1C, a stent 20 is placed over a balloon 12 carried near a distal end of a catheter 14, and is directed through the lumen 16 (FIG. 1A) until the portion carrying the balloon and stent reaches the region of an occlusion 18. The stent 20 is then radially expanded by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C).

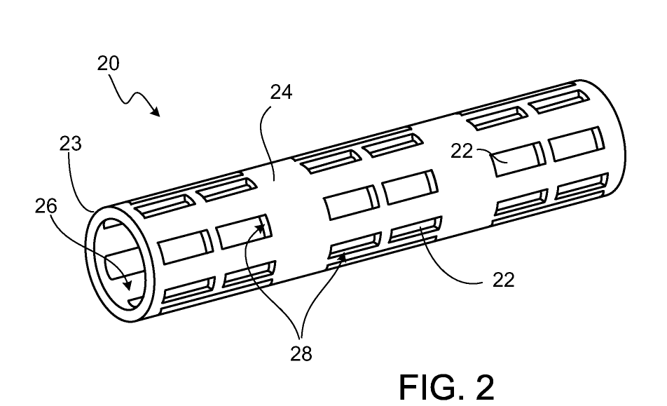

Referring to FIG. 2, stent 20 includes a plurality of fenestrations 22 defined in a wall 23. Stent 20 includes several surface regions, including an outer, or abluminal, surface 24, an inner, luminal (or adluminal), surface 26, and a plurality of cutface surfaces 28. The stent can be balloon expandable, as illustrated above, or a self-expanding stent. Examples of stents are described in Heath '721, supra.

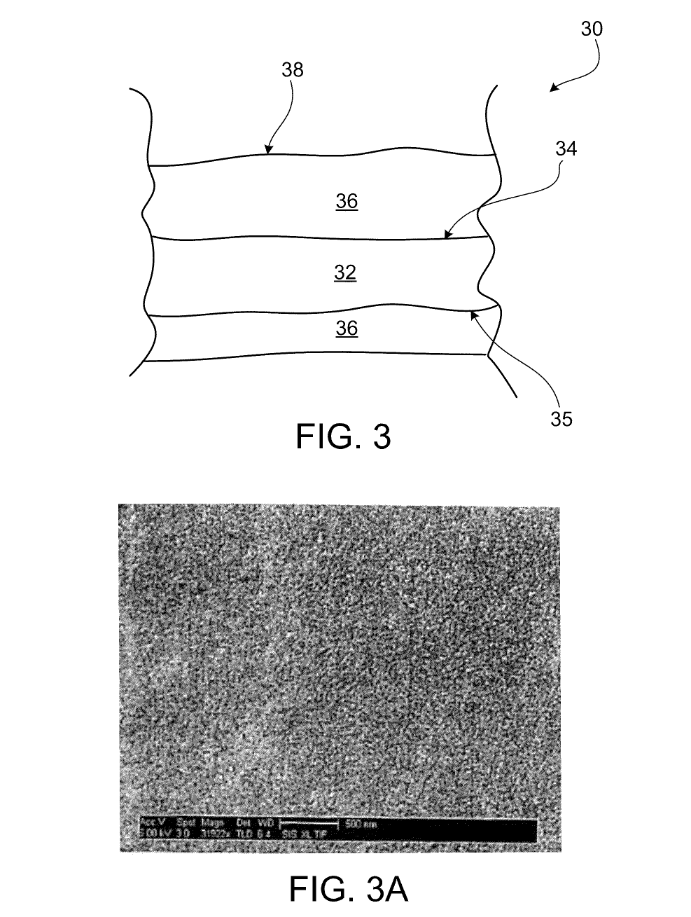

Referring to FIG. 3, a stent wall 30 includes a stent body 32 and a ceramic coating 36 on a surface, e.g., abluminal surface 34 or adluminal surface 35, of stent body 32.

In some embodiments, stent body 32 is formed, e.g., of a metallic material such as a metal or a metal alloy. Examples of the metallic material include 316L stainless steel, Co—Cr alloy, Nitinol, PERSS, MP35N, and other suitable metallic materials.

Ceramic coating 36 includes a ceramic material. Examples of the ceramic material includes iridium oxide (IROX), titanium oxide (TiOₓ), tin oxide (SnOₓ), ruthenium oxide (RuOₓ), tantalum oxide (TaOₓ), niobium oxide (NbOₓ), zirconium oxide (ZrOₓ), cerium oxide (CeOₓ), and tungsten oxide (WOₓ). In some embodiments, in addition to the ceramic material such as IROX, surface 38 of ceramic coating 36 also includes surface hydroxyl groups in the form of, e.g., iridium hydroxide. In some embodiments, surface 38 includes IROX with a molar density of at least, e.g., about 30%, 40%, 50%, 60%, 65%, or 70% and/or up to about 100%, 95%, 90%, 85%, 75%, 60% or 50%, and iridium hydroxide with a molar density of at least, e.g., about 5%, 10%, 15%, or 20% and/or up to about 25%, 30%, 35%, or 40%. In such embodiments, the hydroxyl groups on surface 38 has a molar density of at least, e.g., about 10%, 20%, 30%, or 40% and/or up to about 50%, 60%, 70%, or 80%. The hydroxyl groups are chemically active and can chemically bond an overcoating, such as a polymer coating, to ceramic coating 36 with strong adhesions.

In some embodiments, ceramic coating 36 has a defined rough morphology, such as a rice grain morphology. The rough morphology of ceramic coating 36 can mechanically facilitate interlocking the overcoating on ceramic coating 36. In other embodiments, ceramic coating 36 has a defined smooth morphology, such as an orange peel morphology. Discussion of a rice grain morphology of a ceramic coating is also provided in U.S. patent application Ser. No. 11/752,736, filed May 23, 2007 and U.S. patent application Ser. No. 11/752,772, filed May 23, 2007.

Referring to FIG. 3A, the surface of ceramic coating 36 has an orange peel morphology and is characterized by a continuous surface having a series of globular features separated by striations. The striations have a width of about 10 nm or less, e.g., 1 nm or less, e.g., between 1 nm or about 0.1 nm. The striations can be generally randomly oriented and intersecting. The depth of the striations is about 10% or less of the thickness of the coating, e.g., about 0.1 to 5%.

The orange peel morphology can provide physiological benefits in reducing restenosis and enhancing endothalulyation. In some embodiments, ceramic coating 36 has an orange peel morphology formed conformably about stent body 32 and an overcoating (not shown) formed on ceramic coating 36 only on selected regions, for example, the abluminal region, of stent body 32. When the stent is delivered into a body, the orange peel morphology of ceramic coating 36 on the adluminal side of stent body 32 is exposed to a body lumen and can promote endolithium prohealings. Further, when the overcoating on ceramic coating 36 is biodegradable and degrades away during the use of the stent, the remaining ceramic coating having an orange peel morphology on the abluminal side of the stent in contact with a body lumen may provide similar physiological benefits. In some embodiments, ceramic coating 36 having an orange peel morphology can have a thin thickness, for example, of about 1 nm to about 1 micron. The thin thickness of ceramic coating 36 can prevent the coating from delamination upon expansion of the stent.

Surfaces 34 and 35 can have a roughened nano-structured morphology. For example, the nano-structures on the surfaces can have a size of about 1 nm to about 100 microns. The size and feature of the nano-structured morphology can be controlled by controlling the conditions of forming the morphology. Roughened surfaces 34 and 35 can improve adhesion of ceramic coating 36 to stent body 32 and decrease the possibility of delamination of ceramic coating 36.

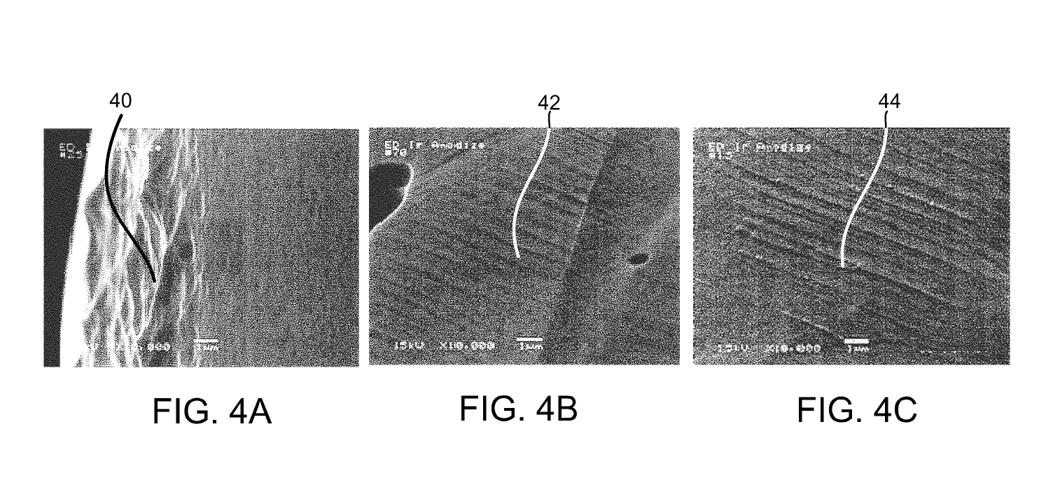

Referring now to FIGS. 4A-4C, stent body 32 coated with ceramic coating 36 undergoes an over expansion that expands stent body 32 to about 2 to about 4 times of its un-expanded size. Ceramic coating 36 has a smooth orange peel morphology. High strain locations 40, 42, and 44 are created on the stent, but no delamination takes place in ceramic coating 36.

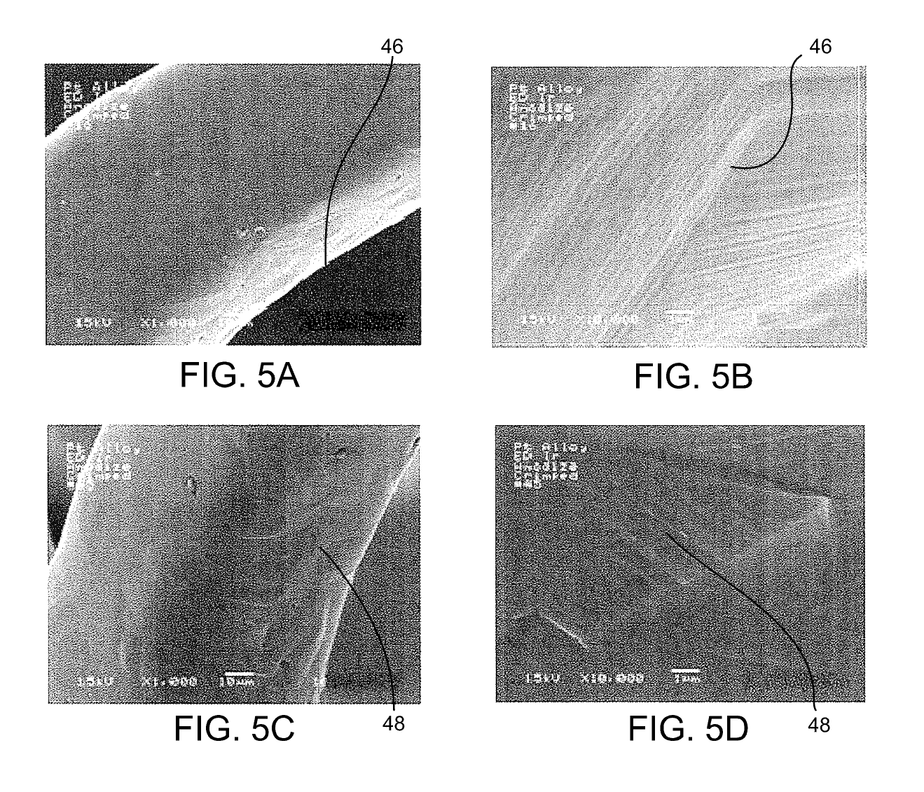

Referring to FIGS. 5A-5D, stent body 32 having ceramic coating 36 is expanded and crimped. The stent walls have similar features, such as coating thickness and morphology, to those in FIGS. 4A-4C. High strain regions 46 and 48 are created on the stent, but no delamination appears in ceramic coating 36.

In some embodiments, an overcoating including a polymer material can be formed on ceramic coating 36. Examples of the polymer material are poly(lactic-co-glycolic acid) (PLGA), and other polymers such as poly(ethylene glycol) (PEG) that can adhere to the ceramic coating 36 through formation of hydrogen bonds. The overcoating can contain a therapeutic agent.

Optionally, a tie layer is disposed between ceramic coating 36 and the overcoating. In some embodiments, the tie layer includes silane and has a thickness, for example, of about 1 nm to about 10 nm (e.g., about 1 nm to about 8 nm or about 2 nm to about 5 nm). The tie layer can further enhance the adhesion between the overcoating and ceramic coating 36. For example, the tie layer can be bonded to the ceramic coating both chemically and mechanically and tie the overcoating with strong adhesions.

In other embodiments, stent wall 30 can also include one or more additional coatings between stent body 32 and ceramic coating 36. For example, one or more coatings including a metallic material, such as Ir, Ru, Ti, Zr, Ta, Nb, Ce, Pt, or Sn, can be disposed between stent body 32 and ceramic coating 36. In such embodiments, a surface of the one or more coatings that contacts ceramic coating 36 preferably has a nano-structured morphology as surface 34. In some embodiments, the additional coating(s) can intermix with ceramic coating 36 and stent body 32 to provide strong adhesion of ceramic coating 36 to stent body 32.

Stent wall 30 having a structure as described above can inhibit delamination of the coatings on stent body 32 and provide good durability. Particularly, the overcoating, such as a polymer coating, containing a therapeutic agent can be tightly bound to stent body 32 to provide desired drug eluting profiles. The peel strength of the overcoating can be affected by the adhesion between ceramic coating 36 and stent body 32 and the adhesion between the overcoating and ceramic coating 36. For example, an enhanced adhesion between ceramic coating 36 and stent body 32 through roughened surfaces 34 and 35 can provide a peel strength of the overcoating up to 10 times as large (for example, 3 to 8 times as large) as that of an overcoating on a stent with an un-roughened surface. For another example, the chemical bonding between the surface hydroxyl group of ceramic coating 36 and the overcoating can increase the peel strength of the overcoating to about 2 to 5 times that without the surface hydroxyl groups. Also, when a tie layer is added between ceramic coating 36 and the overcoating, the peel strength of the overcoating can be up to 10 times larger (e.g., 5 to 8 times larger) than that of the polymer coating on ceramic coating 36 without the tie layer.

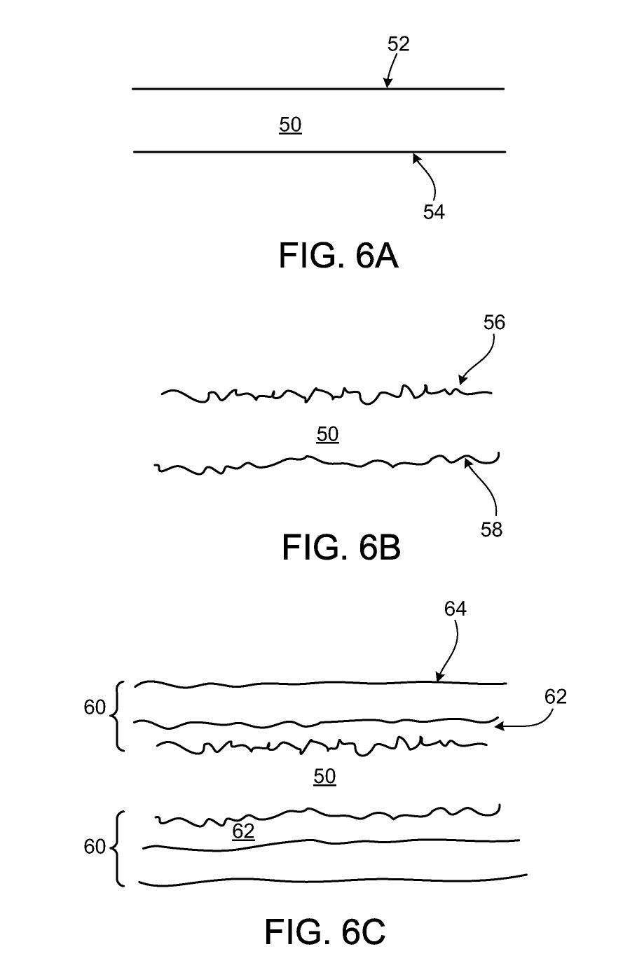

Referring to FIG. 6A, to make a stent exemplified in FIG. 3, a stent preform 50, such as a metal tube, that includes a metallic material is provided. Preform 50 includes various surfaces, e.g., abluminal surface 52 and adluminal surface 54.

Referring now to FIG. 6B, a roughened nano-structured morphology as that of surface 34 is created on the preform surface, e.g., surfaces 52 and 54, by electrolytic etching. For example, preform 50 is treated in an electrolyte solution including an electrolyte, such as phosphoric acid or sulfuric acid. Typically, the electrolyte in the solution has a weight percentage of about 5% to about 99% (e.g. about 20% to about 90% or about 50% to about 70%). In some embodiments, to enhance the effect of the etching, a pulsed waveform is applied on preform 50 to partially recover the consumed electrolyte and produce a highly roughened preform surface. In such embodiments, a pulsed waveform having a positive amplitude, for example of about 200 Ampere/ft² to about 800 Ampere/ft², and a negative amplitude, for example, of about −400 Ampere/ft² to about −1000 Ampere/ft² is applied to preform 50, for example, in an alternative way. The preform surface, such as surfaces 52 and 54 are roughened to be surfaces 56 and 58 having a nano-structured morphology as described above. Generally, the features of the nano-structured morphology created on the preform surfaces are dependent on the type and density of the electrolyte, the time length of the etching, and the conditions of the waveform applied to preform 50, such as frequency and amplitude of the pulses.

In other embodiments, the preform surface, e.g., surfaces 52 and 54, can be roughened by laser irradiation. In still other embodiments, ion bombardment, such as argon ion bombardment, or grit blasting, such as SiC or alumina, can also be used to roughen the preform surface.

In some embodiments, only part of the preform surface, such as surface 52 or part of surface 52 is roughened with the methods discussed above by, e.g., applying selective masking mandrels.

A ceramic coating 60 is conformably electrochemically deposited on the roughened preform surface by first depositing an activation ceramic layer 62 on the roughened preform 50. The electrochemical deposition can be tailored to enhance chemical and metallic bonding between preform 50 and ceramic coating 62.

In some embodiments, an electrolyte, such as hydrogen chloride, and a ceramic precursor, e.g., iridium chloride, are applied to the roughened preform 50. A pulsed waveform having a negative magnitude, e.g., of about −50 mA/cm² to about −10 mA/cm² can be applied on the preform during the deposition. The so-formed activation ceramic layer 62 includes, e.g., IROX. Activation layer 62 is deposited so that a strong mechanical interlocking is formed between the roughened preform surface, e.g., surface 56, and the to be formed ceramic coating 60.

Referring to FIG. 6C, more ceramic material, e.g., IROX, is electrochemically deposited so that ceramic coating 60 is formed. For example, preform 50 coated with activation ceramic layer 62 can be treated in an electrolyte solution containing an electrolyte, such as hydrogen bromide, and a ceramic precursor, such as iridium oxide dihydride. A pulsed waveform having a negative magnitude, e.g., of about −20 mA/cm² to about −1 mA/cm², can be applied to preform 50 during the deposition and ceramic coating 60 is formed on preform 50. The use of electrochemical deposition produces a good intermixing of materials of the activation layer 62, for example, Ir, and materials of the ceramic layer 60, for example, IROX, and enhances the adhesion between the layers to prevent delamination of the ceramic coating 60. In other embodiments, ceramic coating 60 can be selectively formed on part of the roughened preform surface, e.g., surface 56. Electrochemical deposition is also discussed in R. T. Atanasoski et al., J. Electroanal. Chem. 330, 663-673 (1992).

The feature of the so-formed ceramic coating 60 can be modified by cyclic voltammetry. For example, preform 50 with ceramic coating 60 is treated in a sulfuric acid solution, and a pulsed waveform having a positive magnitude, e.g., of about 1 V to about 10 V, and a negative magnitude, e.g., of about −0.1 V to about −1.0 V, is applied to preform 50, e.g., in an alternative way with each magnitude lasting about 10 seconds to 50 seconds. The cyclic voltammetry facilitates forming high surface hydroxyl groups on ceramic coating surface 64. In particular, the oxides in the ceramic coating 64 are protonated by H⁺ and/or OH⁻ within the solution. The density of the formed hydroxyl groups depends on, for example, the thermodynamic properties and the polarization potentials of the coating that vary with the material included in the ceramic coating 64. For example, when the ceramic coating 64 includes TiO₂, the surface density of the hydroxyl group is for example, at least about 1.8×10⁻⁵ mol/m². Detailed discussion of the mechanism of forming hydroxyl groups through electrodeposition is provided in Chang et al., Electrochemical and Solid-State Letters 5, C71-C74 (2002).

In some embodiments, a hydroxylated ceramic layer similar to the ceramic coating 60 can also be formed by converting a metallic layer, for example, an iridium layer deposited, e.g., electro-deposited, on surfaces of preform 50. For example, an iridium hydrobromide acidic bath can be applied to the preform 50 to electro-deposit a layer of iridium on the preform 50. Pulsed electrolytic waveforms or cyclic voltammetry are subsequently applied to the metallic layer. Anhydrous and hydrous layers are alternatively formed and disrupted to generate a ceramic layer having a hydroxylated surface.

In some embodiments, a cleaning process is performed before and after one or more of the preform surface roughening, activation layer deposition, ceramic coating deposition and cyclic voltammetry processes described above. For example, preform 50 is rinsed by deionized water with partial agitation. The cleaning process substantially prevents contaminations carried from different processes, such as different electrolytes, and therefore facilitates producing high quality, for example, robust, coatings on preform 50.

In some embodiments, an additional coating can be formed on an unroughened preform surface, e.g., surfaces 52 and 54, or a roughened preform surface, e.g., surfaces 56 and 58, before the deposition of ceramic layer 60. The surface of the additional coating that contacts ceramic coating 60 can have a roughened nano-structured morphology as that of surface 56. Such morphology can be created in a similar way to those used in the roughening the stent preform described in FIG. 6B. In other embodiments, more than one such additional coatings can be formed between preform 50 and ceramic coating 60.

An optional tie layer including, e.g., silane, can be formed on ceramic coating 60 by self-assembly. For example, a silane coupling agent including a trimethoxy or triethoxy silane can be used to react with and covalently bonded to the oxides in the ceramic coating. Detailed information of forming silane layer on a ceramic coating is provided in Pitt et al., Journal of Biomedical Materials Research Part A Volume 68A, Issue 1, Pages 95-106.

In some embodiments, a polymer coating containing a polymer material and a therapeutic agent is deposited on the ceramic coating by spray coating, dip coating, ink jet printing, or roll coating. In some embodiments, the polymer material can be mixed with a coupling agent, such as silane, before the deposition. In such embodiments, the coupling agent can have a weight percentage of about 1% to about 20% in the polymer coating.

### EXAMPLE 1

In this illustrative example, a stent exemplified in FIG. 3 is made from an endoprosthesis preform using a method including the following procedures.

A stainless steel stent preform is soaked in a Technic 1508 Cleaner (Technic, Inc., Rhode Island) at about 120 F for about 2 minutes with agitation. The stent preform is then rinsed with deionized water at room temperature with partial agitation for about 50 seconds. The rinsed preform is electrolytically etched with agitation at about 25° C. in a solution containing about 70.5 wt % of phosphoric acid, about 5.8 wt % of sulfuric acid, about 4.7 wt % of thiourea, and about 19 wt % of water for about 350 seconds. At the same time, a first pulsed waveform having a positive magnitude of about 545 Ampere/ft² and a second pulsed waveform having a negative magnitude of about −815 Ampere/ft² are alternatively applied on the preform for about 450 ms and about 50 ms, respectively. The etched preform is then rinsed for about 50 seconds with deionized water at room temperature.

To form a first layer of IROX, the etched preform is placed in a solution that contains about 0.5 molar of hydrogen chloride and an iridium chloride having a density of about 5 g/L at room temperature for about 2 minutes. When a cyclic voltammetry or pulsed electrolytic waveforms are applied to the preform, oxides on the surfaces of the preform are removed and an iridium layer is formed on the surfaces. In particular, a pulsed waveform having a magnitude of about −21 mA/cm² is concurrently applied to the preform with agitation. After rinsing for another about 50 seconds with deionized water, at about 167 F, the preform is soaked in a solution containing about 0.1 molar of hydrobromic acid and iridium oxide dihydride at a density of about 6.8 g/L when a pulsed waveform having a magnitude of about −7.1 mA/cm² is periodically applied on the preform for about 3 ms and followed by a 7 ms intermission. After another about 50 seconds of rinsing, a cyclic voltammetry is applied to the ceramic coating on the preform. In particular, a first pulsed waveform having a negative magnitude of about −0.241 V and a second pulsed waveform having a positive magnitude of about 1.26 V are alternatively applied on the preform for about 0.045 second and about 0.080 second, respectively. The process is performed under room temperature in a solution having about 0.5 molar of sulfuric acid for about 12.5 seconds. Finally, the preform coated with ceramic is rinsed and a stent exemplified in FIG. 3 is made.

### EXAMPLE 2

In this illustrative example, four stents coated with a ceramic coating and a polymer coating are made. The peel strength of the polymer coating on each stent is measured.

The first stent is made by depositing a PLGA with 2% of silane onto the stent prepared in Example 1. The stent is then soaked in phosphate buffered saline (PBS) at 37° C. for about 4 days.

The second stent is made by depositing a PLGA with 2% of silane onto the stent prepared in Example 1, except that the before depositing the ceramic coating, the preform is not electrolytically etched, but is instead electropolished and dipped the preform in a sodium hydroxide solution. The stent is then soaked in phosphate buffered saline (PBS) at 37° C. for about 4 days.

The third stent is made by depositing a PLGA with 2% of silane onto the stent prepared in Example 1, except that the before depositing the ceramic coating, the preform is not electrolytically etched, but is instead electropolished and treated with plasma. The stent is then soaked in phosphate buffered saline (PBS) at 37° C. for about 4 days.

The fourth stent is made in the same way as the second stent, except there is no silane included in PLGA.

The peel strength of the PLGA coating on each of the four stents are measured. Results show that the peel strength of the PLGA coating on the first stent is the highest and is about 1000 g/inch. The peel strength of PLGA coating on the second, third, and fourth stent is about 220 g/inch, 180 g/inch, and 50 g/inch, respectively.

The terms “therapeutic agent,” “pharmaceutically active agent,” “pharmaceutically active material,” “pharmaceutically active ingredient,” “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total.

Exemplary therapeutic agents include, e.g., anti-thrombogenic agents (e.g., heparin); anti-proliferative/anti-mitotic agents (e.g., paclitaxel, 5-fluorouracil, cisplatin, vinblastine, vincristine, inhibitors of smooth muscle cell proliferation (e.g., monoclonal antibodies), and thymidine kinase inhibitors); antioxidants; anti-inflammatory agents (e.g., dexamethasone, prednisolone, corticosterone); anesthetic agents (e.g., lidocaine, bupivacaine and ropivacaine); anti-coagulants; antibiotics (e.g., erythromycin, triclosan, cephalosporins, and aminoglycosides); agents that stimulate endothelial cell growth and/or attachment. Therapeutic agents can be nonionic, or they can be anionic and/or cationic in nature. Therapeutic agents can be used singularly, or in combination. Preferred therapeutic agents include inhibitors of restenosis (e.g., paclitaxel), anti-proliferative agents (e.g., cisplatin), and antibiotics (e.g., erythromycin). Additional examples of therapeutic agents are described in U.S. Published Patent Application No. 2005/0216074. In embodiments, the drug can be incorporated within the porous regions in a polymer coating. Polymers for drug elution coatings are also disclosed in U.S. Published Patent Application No. 2005/019265A. A functional molecule, e.g., an organic, drug, polymer, protein, DNA, and similar material can be incorporated into grooves, pits, void spaces, and other features of the stent.

Any stent described herein can be dyed or rendered radiopaque by addition of, e.g., radiopaque materials such as barium sulfate, platinum or gold, or by coating with a radiopaque material. The stent can include (e.g., be manufactured from) metallic materials, such as stainless steel (e.g., 316L, BioDur® 108 (UNS S29108), and 304L stainless steel, and an alloy including stainless steel and 5-60% by weight of one or more radiopaque elements (e.g., Pt, Ir, Au, W) (PERSS®) as described in US-2003-0018380-A1, US-2002-0144757-A1, and US-2003-0077200-A1), Nitinol (a nickel-titanium alloy), cobalt alloys such as Elgiloy, L605 alloys, MP35N, titanium, titanium alloys (e.g., Ti-6Al-4V, Ti-50Ta, Ti-10Ir), platinum, platinum alloys, niobium, niobium alloys (e.g., Nb-1Zr) Co-28Cr-6Mo, tantalum, and tantalum alloys. Other examples of materials are described in commonly assigned U.S. application Ser. No. 10/672,891, filed Sep. 26, 2003; and U.S. application Ser. No. 11/035,316, filed Jan. 3, 2005. Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; and commonly assigned U.S. application Ser. No. 10/346,487, filed Jan. 17, 2003.

The stents described herein can be configured for vascular, e.g., coronary and peripheral vasculature or non-vascular lumens. For example, they can be configured for use in the esophagus or the prostate. Other lumens include biliary lumens, hepatic lumens, pancreatic lumens, urethral lumens.

The stent can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., see U.S. Pat. No. 6,290,721).

Other embodiments are in the following claims.

What is claimed is:

1. A method of making an endoprosthesis comprising:
forming a ceramic coating on a surface of an endoprosthesis wall; and
conducting cyclic voltammetry on the endoprosthesis wall.

2. The method of claim 1, wherein forming the ceramic coating comprises electrochemically depositing a ceramic coating on the surface of the endoprosthesis wall.

3. The method of claim 1, wherein the surface is a nano-structured surface.

4. The method of claim 3, wherein the nano-structured surface is a surface of an endoprosthesis preform.

5. The method of claim 1, wherein the surface of the endoprosthesis preform comprises abluminal, luminal, and cutface surfaces.

6. The method of claim 3, comprising forming the nano-structured surface by laser processing, ion bombardment, grid blasting, or electrolytic etching.

7. The method of claim 3, comprising forming the nano-structured surface by electrolytic etching.

8. The method of claim 3, wherein the nano-structured surface is a surface of a coating between the ceramic coating and an endoprosthesis preform.

9. The method of claim 1, further comprising forming a polymer coating on the ceramic coating.

10. The method of claim 9, further comprising forming a tie layer between the polymer coating and the ceramic coating.

11. The method of claim 1, wherein the ceramic coating comprises IROX.

12. The method of claim 1, wherein the ceramic coating comprises surface hydroxyl groups having a density of at least 1.8×10⁻⁵ mol/m².

13. The method of claim 1, wherein forming the ceramic coating comprises depositing a metallic layer on the surface of the endoprosthesis wall.

14. The method of claim 13, wherein forming the ceramic coating further comprises converting the metallic layer into a ceramic layer.

15. The method of claim 14, wherein the converting comprises applying a cyclic voltammetry to the metallic layer.

16. The method of claim 14, wherein the converting comprises applying pulsed electrolytic waveforms to the metallic layer.

17. The method of claim 13, wherein depositing the metallic layer comprises applying a solution to the endoprosthesis wall.

18. The method of claim 13, wherein the solution comprises an iridium hydrobromide acidic bath.

19. A method of making an endoprosthesis comprising:
forming a metallic coating on a surface of an endoprosthesis wall; and
conducting cyclic voltammetry on the metallic coating to convert the metallic coating into a ceramic coating.

20. The method of claim 19, the ceramic coating comprises a hydroxylated surface.

21. An endoprosthesis comprising:
an endoprosthesis preform having a ceramic coating, the ceramic coating comprising surface hydroxyl groups having a density of at least 1.8×10⁻⁵ mol/m².

22. The endoprosthesis of claim 21, further comprising a coating between the ceramic coating and the preform.

23. The endoprosthesis of claim 22, wherein the coating comprises a nano-structured surface and the ceramic coating is on the nano-structured surface.

24. The endoprosthesis of claim 21, further comprising a polymer coating on the ceramic coating.

25. The endoprosthesis of claim 24, further comprising a tie layer between the ceramic coating and the polymer coating.

26. The endoprosthesis of claim 25, wherein the tie layer comprises silane.
